(19) United States
(12) Patent Application Publication
(10) Pub. No.: US 2019/0080490 A1
(43) Pub. Date: Mar. 14, 2019

(54) REDUCING MOVEMENT ARTIFACTS IN COMPUTED TOMOGRAPHY IMAGE DATA

(71) Applicant: Siemens Healthcare GmbH, Erlangen (DE)

(72) Inventors: Harald SCHOENDUBE, Erlangen (DE); Steffen KAPPLER, Effeltrich (DE)

(73) Assignee: Siemens Healthcare GmbH, Erlangen (DE)

(21) Appl. No.: 16/127,352

(22) Filed: Sep. 11, 2018

(30) Foreign Application Priority Data
Sep. 13, 2017 (EP) 17190895.7

(51) Int. Cl. G06T 11/00; G06T 7/00; A61B 6/03; A61B 6/00

(52) U.S. Cl. CPC G06T 11/005; G06T 7/0012; A61B 6/032; G06T 2207/10116; A61B 6/5264; G06T 2207/30004; A61B 6/4452

(57) ABSTRACT

A method is for imaging a region of interest of an object to be examined based on projection measurement data recorded during a rotational movement of an X-ray source-detector system around the object to be examined in a first angular sector of at least 180° . In an embodiment, the method includes generating first start-image data; selecting partial projection measurement data with a second angular sector from the projection measurement data, the second angular sector being a subregion of the first angular sector; comparing the first start-image data generated, or corrected start-image data, with the partial projection measurement data, and generating first image data based upon the comparing; and artifact correcting the first image data via a trained correction unit, to create the corrected start-image data, the first image data and the corrected start-image data each respectively comprising a substantially complete image of the region of interest.

### PRIORITY STATEMENT

The present application hereby claims priority under 35 U.S.C. § 119 to European patent application number EP17190895.7 filed Sep. 13, 2017, the entire contents of which are hereby incorporated herein by reference.

### FIELD

At least one embodiment of the invention generally relates to a method for imaging, an image reconstruction device, a computed tomography system, a computer program and computer-readable data medium, which enable the reduction of movement artifacts in computed tomography image data.

### BACKGROUND

Computed tomography is an imaging method, which is primarily used for medical diagnostics and for examination of materials. In computed tomography, a radiation source, for example an X-ray source, and a detector apparatus interacting therewith rotate about an object to be examined in order to record spatially three-dimensional image data. During the rotational movement, measurement data is recorded within an angular sector. The projection measurement data entails a plurality of projections containing information about the attenuation of the radiation by the examination object from different projection angles. A two-dimensional image slice or a three-dimensional volume image of the examination object can be calculated from these projections. The projection measurement data is also referred to as raw data or the projection measurement data can already be preprocessed in order, for example, to reduce detector-induced attenuation intensity differences. Image data can then be reconstructed from this projection measurement data, for example by use of so-called filtered back projection or by use of an iterative reconstruction method. If, for example, the examination object moves during the recording, unsharp areas and artifacts resulting from the movement can occur during the reconstruction of the image data.

Multifarious methods for scanning an examination object with a computed tomography system are known. For example, orbital scans, sequential orbital scans with advance or spiral scans are employed. Other types of scan, which are not based on orbital movements, are also possible, for example scans with linear segments. Absorption data of the examination object is recorded from different recording angles with the aid of at least one X-ray source and at least one opposing detector apparatus and the absorption data or projection measurement data collected in this way is computed into image slices through the examination object by way of corresponding reconstruction methods.

For the reconstruction of computed tomography images from the projection measurement data of a computed tomography system, the standard method used nowadays is known as the filtered back projection method (FBP) or iterative reconstruction. Due to their approximative method of operation, problems can occur with filtered back projection methods with so-called cone beam artifacts, spiral artifacts, spiral artifacts and limited-view artifacts. The filtered back projection method belongs to the group of approximative reconstruction methods. There is also a group of exact reconstruction methods in existence, although this is hardly ever used nowadays. Iterative methods form a third group of reconstruction method.

Iterative reconstruction methods enable at least some of the aforementioned limitations of filtered back projection to be overcome. Such an iterative reconstruction method starts with the reconstruction of start-image data from the projection measurement data. A filtered back projection method can be used for this purpose, for example. The iterative reconstruction method subsequently gradually generates improved image data. For example, synthetic projection data can be generated from the start-image data with a “projector”, a projection operator that is designed to map the measurement system mathematically as well as possible. The difference from the measurement signals is then projected back with the operator adjoining the projector and, in this way, a residual image is reconstructed with which the initial image is updated. The updated image data can in turn be used, in a next iteration step with the aid of the projection operator, to generate new synthetic projection data, to form the difference from the measurement signals therefrom once again and to calculate a new residual image with which image data of the present iteration stage is again improved. Examples of iterative reconstruction methods are the algebraic reconstruction technique (ART), the simultaneous algebraic reconstruction technique (SART), iterated filtered back projection (IFBP), or also statistical iterative image reconstruction techniques.

The publication DE 10 2006 007 058 A1 discloses a CT system with a gantry for fastening at least one focus/detector system that is rotatable about a system axis. The CT system comprises a focus as a radiation source and a detector that receives radiation emitted by the focus, a first focus/detector system and at least one second focus/detector system arranged rotationally offset to the first, which is mechanically coupled to the first focus/detector system and co-rotates therewith on the gantry, wherein each focus/detector system comprises a central beam that extends from the focus through the system axis to the detector and a mid-beam that extends from the focus to the geometrical center of the detector and at least one focus/detector system is arranged eccentrically with respect to the system axis.

The publication DE 10 2008 051 043 B3 discloses an X-ray CT system and a method for producing tomographic recordings with the aid of an X-ray CT system with two emitter-detector arrangements operating with an angular offset on a gantry with at least two different X-ray energy spectra, wherein at least one first recording is reconstructed from two quarter rotations with different X-ray energy spectra and at least one second recording is created from detector data of a scan of at least one of the emitter-detector arrangements over a half rotation. The recordings are subject to high-pass filtering or low-pass filtering in respect of their spatial frequencies and then the filtered recordings are combined to form a result recording.

The publication DE 10 2010 019 016 A1 discloses a method for the reconstruction of image data of a moving examination object from measurement data, wherein the measurement data has previously been established in a relative rotational movement between a radiation source of a computed tomography system and the examination object. First image data is reconstructed from an incomplete measurement data record by use of an iterative algorithm, wherein in the iterative reconstruction, a dimension is used that contains probability information relating to pixel values of the image data to be reconstructed.

Two emitter-detector arrangements offset by 90° can be used for simultaneous recording of data so that a projection angle coverage of 180° is already attained after a gantry rotation of 90°. This corresponds to a doubling of the achievable time resolution. Alternatively, the recording can be made over two temporal sections of 90° each, wherein in each case they depict the same movement status of the heart over successive heartbeats. The maximum possible time resolution is doubled. The achievable time resolution can also be increased by increasing the rotational speed of the gantry. However, the mechanical solution to the problem of limited time resolution is subject to constraints.

In computed tomography, the time resolution of an in particular single image (slice) is defined by the time period in which the individual projections or the projection measurement data of an angular sector from which the image was reconstructed were recorded. Therefore, for applications in which time resolution is critical, for example in coronary imaging, typically only the minimum necessary number of projections or projection measurement data of a minimum necessary angular sector is used.

For hitherto common image reconstruction techniques, generally a minimum necessary projection angle range of 180° is assumed. In the case of a rotational speed of the gantry, which is included in the image recording system consisting of an X-ray source and a detector apparatus, of 0.5 seconds, corresponding to the minimum necessary angular sector of 180°, a maximum time resolution of 0.25 seconds can be achieved since at least the projection data from half a revolution is required.

### SUMMARY

The inventors have discovered that if the region of interest of the examination object to be represented during the recording of the projections or projection measurement data moves, for example, out of the minimum necessary angular sector, so-called movement artifacts form in the reconstructed (result) image. The reduction or elimination of these movement artifacts is achieved by at least one embodiment of the present invention.

At least one embodiment of the invention provides a method for imaging, an image reconstruction device, a computed tomography system, a computer program and a computer-readable data medium that facilitate the reduction of movement artifacts or an increase in time resolution.

Embodiments of the invention is directed to a method, an image reconstruction device, a computed tomography system, a computer program and a computer-readable data medium.

At least one embodiment of the invention relates to a method for imaging a region of interest of an object to be examined based on projection measurement data recorded during a rotational movement of an X-ray source-detector system around the object to be examined in a first angular sector of at least 180°. The method comprises the steps of generation, selection, comparison and artifact correction. In the generation step, first start-image data is generated. In the selection step, partial projection measurement data is selected with a second angular sector from the projection measurement data, wherein the second angular sector is a subregion of the first angular sector. In the comparison step, the first start-image data or corrected start-image data is compared with the partial projection measurement data, wherein first image data is generated. In the artifact correction step, the first image data is corrected via a trained correction unit, wherein the corrected start-image data is created. The first image data and the corrected start-image data in each case comprise a substantially complete image of the region of interest.

At least one embodiment of the invention further relates to an image reconstruction device for performing the method according to at least one embodiment of the invention for imaging, including at least one processor for example. The image reconstruction device of at least one embodiment can also include a generating unit for generating first start-image data, a selecting unit for selecting partial projection measurement data with a second angular sector from the projection measurement data, wherein the second angular sector is a subregion of the first angular sector, a comparing unit for comparing the first start-image data or corrected start-image data with the partial projection measurement data, wherein first image data is generated, and an artifact correction unit for artifact correction of the first image data via a trained correction unit, wherein the corrected start-image data is created. It is advantageously possible for the method according to the invention to be performed in an image reconstruction device. The image reconstruction device can be included in a computing unit. The image reconstruction device or the computing unit can be included in the computed tomography system. The image reconstruction device can, for example, use projection measurement data or start-image data from cloud storage to perform the method.

At least one embodiment of the invention further relates to a computed tomography system with an image reconstruction device according to at least one embodiment of the invention. It is advantageously possible for the method for image generation or for artifact correction to be performed directly in the computed tomography system. It is advantageously possible for a result image to be created from the projection measurement data and displayed to the user.

At least one embodiment of the invention further relates to a computer program with program code for performing the method according to at least one embodiment of the invention when the computer program is executed on a computer.

At least one embodiment of the invention further relates to a non-transitory computer-readable data medium with program code of a computer program for performing the method according to at least one embodiment of the invention when the computer program is executed on a computer.

At least one embodiment of the invention further relates to a method for training the correction unit comprising the generation and training steps. In the generation step, training data is generated. The training data can include first start-image data and projection measurement data. The training data can be generated via a computed tomography system. The training data can be generated artificially, for example by simulation. The method for training can comprise a selection step, wherein partial projection measurement data with a second angular sector is selected from the projection measurement data, wherein the second angular sector is a subregion of the first angular sector. The result image can be predetermined. The predetermined result image can be an artifact-reduced or artifact-free result image. The method for training can further comprise a comparison step, wherein the first start-image data or corrected start-image data is compared with the partial projection measurement data, wherein first image data is generated. The method for training can further comprise an artifact correction step, wherein the first image data is corrected via a trained correction unit, wherein the corrected start-image data is created. The first image data and the corrected start-image data can in each case comprise a complete image of the region of interest.

### BRIEF DESCRIPTION OF THE DRAWINGS

The following explains example embodiments of the invention in more detail with reference to drawings, which show:

FIG. 1 a schematic representation of the method according to the invention for imaging according to a first embodiment;

FIG. 2 a schematic representation of the method according to the invention for imaging according to a second embodiment;

FIG. 3 a schematic representation of the method according to the invention for imaging according to a third embodiment;

FIG. 4 a schematic representation of the method according to an embodiment of the invention for training the correction unit; and

FIG. 5 a schematic concept of a computed tomography system according to an embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated.The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or porcessors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a method for imaging a region of interest of an object to be examined based on projection measurement data recorded during a rotational movement of an X-ray source-detector system around the object to be examined in a first angular sector of at least 180°. The method comprises the steps of generation, selection, comparison and artifact correction. In the generation step, first start-image data is generated. In the selection step, partial projection measurement data is selected with a second angular sector from the projection measurement data, wherein the second angular sector is a subregion of the first angular sector. In the comparison step, the first start-image data or corrected start-image data is compared with the partial projection measurement data, wherein first image data is generated. In the artifact correction step, the first image data is corrected via a trained correction unit, wherein the corrected start-image data is created. The first image data and the corrected start-image data in each case comprise a substantially complete image of the region of interest.

In at least one embodiment of the invention,a result image is reconstructed from computed tomographic projection measurement data, wherein the time resolution to be achieved is less than the time period within which projection data is scanned in a first angular sector of, for example, at least 180°. It is advantageously possible to avoid or reduce so-called “limited angle” artifacts, which occur, for example, with the direct application of known reconstruction methods, for example filtered back projection (FBP). The first image data and the corrected start-image data in each case comprise a substantially complete image of the region of interest. For example, the region of interest can, for example, be restricted to a subregion of a image slice or a recording region. The region of interest can, for example, comprise only one limited subregion including a predetermined anatomical subregion of the object to be examined or the region of interest. The region of interest can for example substantially include only one single organ, for example the heart, and possibly additionally a marginal region of the tissue surrounding the organ.

Compared to the first start-image data, the substantially complete image can in particular comprise reduced “limited angle” artifacts or no “limited angle” artifacts. Compared to the start image, the substantially complete image can in particular comprise reduced so-called directional artifacts or “limited angle” artifacts, which can be induced by the use of the second angular sector of in particular less than 180°. The substantially complete image can, for example, comprise substantially correct image information of the region of interest.

On the other hand, the first start-image data can include “limited angle” artifacts or directional artifacts. So-called “limited angle” artifacts can in particular occur as a result of the use of an over-small, for example second, angular sector in the reconstruction. For example, if an object with a substantially circular cross section is observed in the plane of the image slice in the region of interest, as a rule, the use of the second angular region in the reconstruction, for example by use of filtered back projection, produces, instead of a circular cross section, an onion-shaped or almond-shaped cross section, wherein the two peaks of the onion-shaped or almond-shaped cross section point in the direction of the central projection within the second angular sector. In particular in the difference area of the onion-shaped or almond-shaped cross section and of the circular cross section, the image can be incomplete, i.e. in this difference area, the information on the object is reproduced incompletely or incorrectly. In the substantially complete image, the object with a substantially circular cross section can also have a circular cross section.

The method according to at least one embodiment of the invention uses a subregion of the projection measurement data, for example 120°, for example in conjunction with a boundary condition, which is based on a database that can be based on a dictionary learning principle.

The projection measurement data can be present or generated in the form of a sinogram covering or including a first angular sector of at least 180°. Then, in particular, a subregion of the sinogram is selected, wherein the subregion includes a second angular sector which is in particular smaller than the first angular sector and is in particular less than 180°.

The first image data or the result image can be reconstructed proceeding from a start image by way of an iterative method in which in particular the comparison and artifact correction steps alternate. In the comparison step, the start-image data is compared with the partial projection measurement data. The result can be stored as first image data. In the artifact correction step, the first image data can, for example, be corrected via a database based on the dictionary-learning principle, wherein the “limited angle” artifacts present in the first image data and any movement artifacts, for example in particular in the region of the coronary arteries, are to be removed or corrected. Herein, the database can, for example, be implemented in the form of an electronic anatomical atlas. For example, an artifact-affected image region of the first image can be corrected via an artifact-free anatomically correct image region held in the database. The result is stored as corrected start-image data. The corrected start-image data can then be used in the comparison step.

The comparison step can in particular advantageously be used to keep the start-image data consistent with the partial projection measurement data. In the artifact correction step, it is in particular possible to remove or reduce artifacts expected as a result of object movement or the use of the second angular sector that is smaller than the first angular sector.

The method according to at least one embodiment of the invention can in particular include the use of a database based on the machine-learning principle in combination with an iterative reconstruction algorithm for image reconstruction using partial projection measurement data of a second angular sector with less than 180°. It is advantageously possible for a result image or a subregion of a region of interest to be reconstructed based on partial projection measurement data of a second angular sector of less than 180°. It is advantageously possible to achieve increased time resolution of the result image.

According to one embodiment of the invention, the trained correction unit is based on a machine learning method, a statistical method, a mapping protocol, mathematical functions, an artificial neural network or a learning database. According to one embodiment of the invention, the trained correction unit uses a machine learning method, a statistical method, a mapping protocol, mathematical functions, an artificial neural network or a learning database. The learning database can also be called a dictionary-learning-based database. The trained correction unit can preferably use a learning database. Advantageously, patterns or regularities from the training data can be applied to the first image data, wherein the corrected start-image data is created. The correction unit can use combinations or weightings of features or parameters in the training data for artifact correction.

Machine learning methods can be described as the artificial generation of knowledge from experience. An artificial system learns from examples in a training phase and is able to generalize after completion of the training phase. This enables the identification unit to be adapted. The use of machine learning methods can include the recognition of patterns and regularities in the training data. After the training phase, the identification unit can, for example, extract features or parameters in previously unknown image data. After the training phase, the identification unit is able to identify a type of artifact, for example based on previously unknown image data. It is advantageously possible to derive a reliable method for artifact identification from knowledge of known training data. Advantageously, the identification unit's experience of samples or regularities can be used as the basis for the identification of an artifact or a type of artifact.

According to one embodiment of the invention, in the generation step, the start-image data is generated based on the projection measurement data. The start-image data can be generated in different ways. The start-image data can for example, be generated by use of filtered back projection of the projection measurement data. The start-image data can, for example, be generated by use of filtered back projection of the partial projection measurement data with a constraint, wherein the constraint can, for example, be based on a database based on a dictionary-learning principle. It is advantageously possible to utilize the similarity of the start-image data to the result image. It is advantageously possible to reduce the number of iteration steps.

According to one embodiment of the invention, in the generation step, the start-image data is generated independently of the projection measurement data. The start-image data can include an empty image or a predetermined image. The predetermined image can, for example, include an artificially generated image. The predetermined image can, for example, be based on a known image in a recording of another examination object. Advantageously, the start-image data can be substantially unaffected by the artifacts.

According to one embodiment of the invention, the comparison and artifact correction steps are performed iteratively, wherein in a first pass, the first start-image data is compared with the partial projection measurement data and, in at least one further pass, the corrected start-image data is compared with the partial projection measurement data. The method can be performed iteratively in several passes until a termination condition is reached. The termination condition can, for example, include a parameter of the comparison or a parameter of the artifact correction. The parameter can, for example, specify that the input data is substantially not further changed in the comparison or artifact correction step. Advantageously, convergence can be achieved gradually in several passes.

According to one embodiment of the invention, the comparison step is performed in the image space. The first start-image data or the corrected start-image data is compared with the partial projection measurement data, wherein the comparison is based on image data of the partial projection measurement data. The image data of the partial projection measurement data can be generated by use of filtered back projection. The comparison can be performed in the image space. It is advantageously possible to avoid repeated forward and backward projections. It is advantageously possible for the method according to an embodiment of the invention to be accelerated.

According to one embodiment of the invention, the comparison step is performed in the projection data space. Forward projection of the corrected start-image data can be performed before the comparison step. The comparison step can include backward projection so that the first image data is formed. The method according to the invention can include a change between the image space and projection data space. It is advantageously possible to achieve greater conformity between the result image and the projection measurement data.

According to one embodiment of the invention, the method further comprises a regularization step. The regularization can include modeling of the, in particular local, noise in the CT image. The regularization can include the separation of information and noise based on statistical significance. It is possible to achieve an advantageous contrast-to-noise ratio in the result image. The regularization can include the subtraction of the noise component ascertained. It is advantageously possible to ensure convergence of the iterative artifact correction. It is advantageously possible to achieve noise reduction or dose reduction.

According to one embodiment of the invention, the second angular sector covers an angle of less than 180°. According to one embodiment of the invention, the second angular sector covers an angle in the range of 100°0 to 140°. The second angular sector can preferably cover 110° to 130°. The second angular sector can in particular preferably cover about 120°. It is advantageously possible to achieve increased time resolution.

According to one embodiment of the invention, the method according to the invention further comprises the step of outputting the first image data or the corrected start-image data as a result image. The first image data or the corrected start-image data can be used as a result image. The respective step in which the result image is produced in the form of the first image data or the corrected start-image data can have a greater weight for the creation or outputting of the result image than the other step. It is advantageously possible to select the weight or the influence of the comparison or artifact correction step by selecting the first image data or the corrected start-image data. The result image can be output to the user or further processed by further steps.

According to one embodiment of the invention, in the artifact correction step, a predetermined anatomical subregion of the object to be examined is corrected in the first image data. The examination object can preferably be a patient. The predetermined anatomical subregion can be included in the heart. The predetermined anatomical subregion can, for example, include a coronary artery. The predetermined subregion can, for example, include only at least one coronary artery. The predetermined anatomical subregion can in particular include subregions of the region of interest required for diagnosis. It is advantageously possible for a database comprising an anatomical atlas to include the predetermined anatomical subregion. It is advantageously possible to achieve a particularly good reduction of artifacts in the predetermined anatomical subregion.

At least one embodiment of the invention further relates to an image reconstruction device for performing the method according to at least one embodiment of the invention for imaging, including at least one processor for example. The image reconstruction device of at least one embodiment can also include a generating unit for generating first start-image data, a selecting unit for selecting partial projection measurement data with a second angular sector from the projection measurement data, wherein the second angular sector is a subregion of the first angular sector, a comparing unit for comparing the first start-image data or corrected start-image data with the partial projection measurement data, wherein first image data is generated, and an artifact correction unit for artifact correction of the first image data via a trained correction unit, wherein the corrected start-image data is created. It is advantageously possible for the method according to the invention to be performed in an image reconstruction device. The image reconstruction device can be included in a computing unit. The image reconstruction device or the computing unit can be included in the computed tomography system. The image reconstruction device can, for example, use projection measurement data or start-image data from cloud storage to perform the method.

At least one embodiment of the invention further relates to a computed tomography system with an image reconstruction device according to at least one embodiment of the invention. It is advantageously possible for the method for image generation or for artifact correction to be performed directly in the computed tomography system. It is advantageously possible for a result image to be created from the projection measurement data and displayed to the user.

At least one embodiment of the invention further relates to a computer program with program code for performing the method according to at least one embodiment of the invention when the computer program is executed on a computer.

At least one embodiment of the invention further relates to a non-transitory computer-readable data medium with program code of a computer program for performing the method according to at least one embodiment of the invention when the computer program is executed on a computer.

At least one embodiment of the invention further relates to a method for training the correction unit comprising the generation and training steps. In the generation step, training data is generated. The training data can include first start-image data and projection measurement data. The training data can be generated via a computed tomography system. The training data can be generated artificially, for example by simulation. The method for training can comprise a selection step, wherein partial projection measurement data with a second angular sector is selected from the projection measurement data, wherein the second angular sector is a subregion of the first angular sector. The result image can be predetermined. The predetermined result image can be an artifact-reduced or artifact-free result image. The method for training can further comprise a comparison step, wherein the first start-image data or corrected start-image data is compared with the partial projection measurement data, wherein first image data is generated. The method for training can further comprise an artifact correction step, wherein the first image data is corrected via a trained correction unit, wherein the corrected start-image data is created. The first image data and the corrected start-image data can in each case comprise a complete image of the region of interest.

In the training step, the identification unit is trained based on the training data. In the training or adaptation step, the correction unit can be adapted based on the training data. The training step can in particular include a machine learning method, wherein it is also possible for a statistical method, a mapping protocol or an artificial neural network to be included. The statistical method can, for example, include fuzzy logic, a self-organizing map, resampling, pattern identification or a support vector machine.

The machine learning method can include aspects of data mining. The machine learning method can include a symbolic system or a subsymbolic system, for example an artificial neural network with or without regression. The machine learning can include monitored, partially monitored, unmonitored, reinforcement or active learning. The machine learning method can include batch learning with which all training data is present concurrently and, for example after processing of all of the training data, patterns and regularities are learned by the correction unit.

Machine learning can include a continuous, incremental or sequential learning method, wherein the patterns and regularities are developed in a temporally offset manner. In the case of continuous, incremental or sequential learning methods, the training data can be lost after one-time execution and adaptation of weightings, for example. In the case of batch learning or in the case of continuous, incremental or sequential learning methods, the training data can be available in stored format and the training data can be repeatedly accessible. The machine learning method can, for example, include deep learning methods or shallow learning methods.

It is advantageously possible to apply knowledge from known training data to unknown partial projection measurement data. It is advantageously possible for the correction unit to enable reliable artifact correction due to training. Projection measurement data from the method for imaging can also be used to train the correction unit, for example in order to improve statistical probabilities of the occurrence of features or parameters due to an increasingly large database. The training method can be performed via a training unit. The training unit can be included in a computing unit.

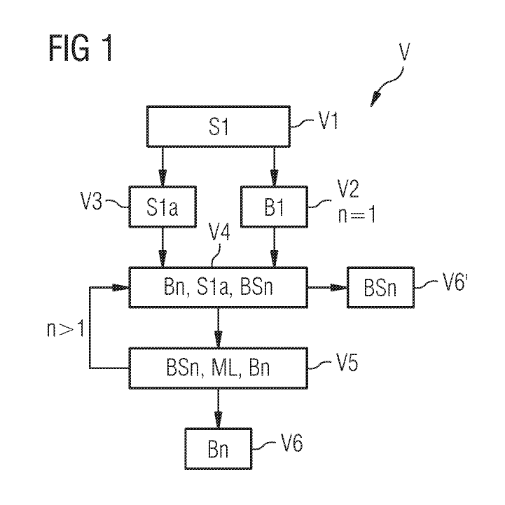

FIG. 1 shows an example embodiment of the method V according to the invention for imaging according to a first embodiment. The method V for imaging a region of interest of an object to be examined is based on projection measurement data S1 recorded during a rotational movement of an X-ray source-detector system around the object to be examined in a first angular sector of at least 18020 . The projection measurement data S1 can be recorded in a first recording step V1.

The method V comprises the generation step V2, the selection step V3, the comparison step V4 and the artifact correction step V5. In the generation step V2, first start-image data B1 is generated. In the selection step V3, partial projection measurement data S1a with a second angular sector is selected from the projection measurement data S1, wherein the second angular sector is a subregion of the first angular sector. The second angular sector covers an angle of less than 180°. The second angular sector preferably covers an angle in the range of 100° to 140°, in particular preferably 120°.

In the comparison step V4, the first start-image data B1 or the corrected start-image data Bn is compared with the partial projection measurement data S1a, wherein first image data BSn is generated. In the artifact correction step V5, the first image data BSn is corrected via a trained correction unit ML, wherein the (new) corrected start-image data Bn is created. The first image data BSn and the corrected start-image data Bn in each case comprise a substantially complete image of the region of interest.

In the outputting step V6, V6′, the first image data BSn or the corrected start-image data Bn can be output as a result image. The trained correction unit ML is based on a machine learning method, a statistical method, a mapping protocol, mathematical functions, an artificial neural network or a learning database.

In the generation step V2, the start-image data B1 is generated based on the projection measurement data S1. It is alternatively possible, in the generation step V2, for the start-image data B1 to be generated independently of the projection measurement data S1. The comparison step V4 and the artifact correction step V5 are performed iteratively, wherein, in a first pass (n=1), the first start-image data B1 is compared with the partial projection measurement data S1a and, in at least one further pass (n>1), the corrected start-image data Bn is compared with the partial projection measurement data S1a. In the artifact correction step V5, it is preferably possible for a predetermined anatomical subregion of the object to be examined in the first image data BSn to be corrected.

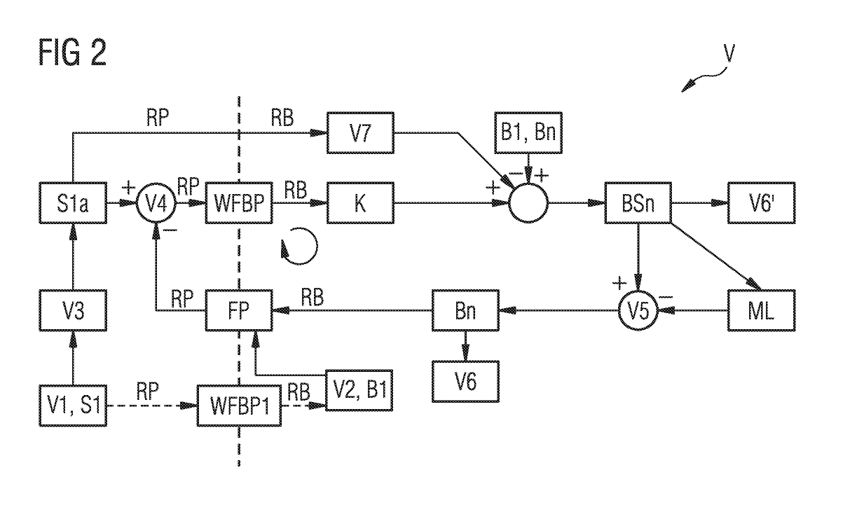

FIG. 2 shows an example embodiment of the method according to the invention for imaging according to a second embodiment. In the generation step V2, first start-image data B1 is generated based on the projection measurement data S1 by use of weighted filtered back projection WFBP1. Herein, there is a transition from the projection data space RP to the image space RB. In the representation, the dashed line separates the projection data space RP and the image space RB.

It is alternatively possible, in the generation step V2, for the start-image data B1 to be generated independently of the projection measurement data S1. In the first pass, the first start-image data B1 is compared with the partial projection measurement data S1a. Herein, the start-image data B1 can be transferred back into the projection data space RP by use of forward projection FP so that the comparison step V4 is performed in the projection data space.

Corrected image data K is generated in that a difference data record is formed from the partial projection measurement data S1a and the forward projection FP of the start-image data B1. Corrected image data K can be created from the difference data record by use of weighted filtered back projection WFBP. The corrected image data K can be used in the image space RB in order to generate first image data BSn by addition of the correction image data K and the start-image data B1.

In the artifact correction step V5, the first image data BSn is corrected via a trained correction unit ML, wherein the corrected start-image data Bn is created. The trained correction unit ML obtains the first image data BSn as an input value. The trained correction unit ML uses the first image data BSn as the basis for determining an artifact correction component. The artifact correction component and the first image data BSn are added together to form corrected start-image data Bn.

In the further passes, the corrected start-image data Bn is transferred back into the projection data space RP by use of forward projection FP so that the comparison step V4 is performed in the projection data space RP. In the further passes, the corrected start-image data Bn is compared with the partial projection measurement data S1a. Herein, the corrected start-image data Bn can be transferred back into in the projection data space RP by use of forward projection FP so that the comparison step V4 is performed in the projection data space RP. Correction image data K can be generated in that a difference data record is formed from the partial projection measurement data S1a and the forward projection FP of the corrected start-image data Bn. Correction image data K can be created from the difference data record by use of weighted filtered back projection WFBP. The correction image data K can be used in the image space RB in order to generate first image data BSn by addition of the correction image data K and the corrected start-image data Bn.

The method can further include a regularization step V7, wherein the regularization V7 is based on the partial projection measurement data S1a and taken into account in the summation of the correction image data K and the start-image data B1 or the corrected start-image data Bn.

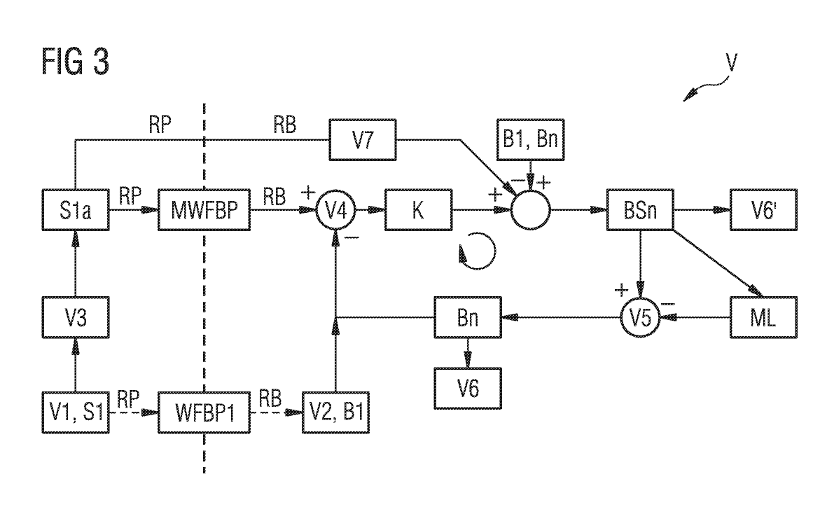

FIG. 3 shows an example embodiment of the method according to the invention for imaging according to a third embodiment. In the generation step V2, first start-image data B1 is generated based on the projection measurement data S1 by use of weighted filtered back projection WFBP1. It is alternatively possible, in the generation step V2, for the start-image data B1 to be generated independently of the projection measurement data S1. In the first pass, the first start-image data B1 is compared with the partial projection measurement data S1a, wherein partial projection measurement data S1a is transferred into the image space RB by use of one-time weighted filtered back projection MWFBP so that the comparison step V4 is performed in the image space RB. Correction image data K can be generated in that a difference data record is formed from the one-time weighted filtered back projection of the partial projection measurement data S1a and the start-image data B1. The difference data record corresponds to the correction image data K. The correction image data K can be used to generate first image data BSn by addition of the correction image data K and the start-image data B1. In the artifact correction step V5, the first image data BSn is corrected via a trained correction unit ML, wherein the corrected start-image data Bn is created. the trained correction unit ML receives the first image data BSn as an input variable. The trained correction unit ML determines an artifact correction component based on the first image data BSn. The artifact correction component and the first image data BSn are added to form corrected start-image data Bn.

In the further passes, the corrected start-image data Bn is compared with the partial projection measurement data S1a, wherein the one-time weighted filtered back projection MWFBP of partial projection measurement data S1a is used again in the further passes without repeating the weighted filtered back projection of the partial projection measurement data S1a. Correction image data K can be generated in that a difference data record is formed from the one-time weighted filtered back projection of the partial projection measurement data S1a and the corrected start-image data Bn. The difference data record corresponds to the correction image data K. The correction image data K is used to generate first image data BSn by addition of the correction image data K and the corrected start-image data Bn.

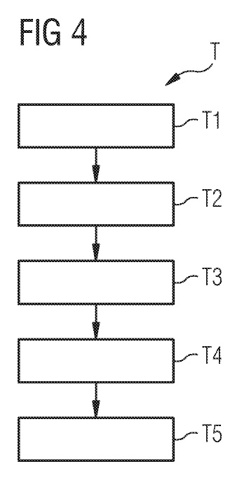

FIG. 4 shows an example embodiment of the method according to the invention for training the correction unit. An example embodiment of the invention further relates to a method T for training the correction unit comprising the generation step T1 and the training step T5.

In the generation step T1, training data is generated. The training data includes, for example, first start-image data and projection measurement data. The training data can be generated via a computed tomography system. Alternatively, the training data can be generated artificially, for example by simulation.

The method for training T can comprise a selection step T2, wherein partial projection measurement data with a second angular sector is selected from the projection measurement data, wherein the second angular sector is a subregion of the first angular sector. The result image can be predetermined. The predetermined result image can be an artifact-reduced or artifact-free result image.

The method for training T can further comprise a comparison step T3, wherein the first start-image data or corrected start-image data is compared with the partial projection measurement data, wherein first image data is generated. The method for training T can further comprise an artifact correction step T4, wherein the first image data is corrected via a trained correction unit, wherein the corrected start-image data is created. The first image data and the corrected start-image data can each comprise a complete image of the region of interest.

In the training step T5, the identification unit is trained based on the training data. In the training step T5 or adaptation step, the correction unit can be adapted based on the training data. The training step T5 can in particular include a machine learning method, wherein it can also include a statistical method, a mapping protocol or an artificial neural network.

The statistical method includes, for example, fuzzy logic, a self-organizing map, resampling, pattern identification or a support vector machine. The machine learning method can include aspects of data mining. The machine learning method can include a symbolic system or a subsymbolic system, for example an artificial neural network with or without regression. The machine learning can include monitored, partially monitored, unmonitored, reinforcement or active learning.

The machine learning method can include batch learning with which all training data is present concurrently and, for example after processing of all of the training data, patterns and regularities are learned by the correction unit. Machine learning can include a continuous, incremental or sequential learning method, wherein the patterns and regularities are developed in a temporally offset manner. In the case of continuous, incremental or sequential learning methods, the training data can be lost after one-time execution and adaptation of weightings, for example.

In the case of batch learning or in the case of continuous, incremental or sequential learning methods, the training data can be available in stored form and the training data can be repeatedly accessible. The machine learning method can, for example, include deep learning methods or shallow learning methods. The training method is performed via a training unit. The training unit is, for example, included in a computing unit.

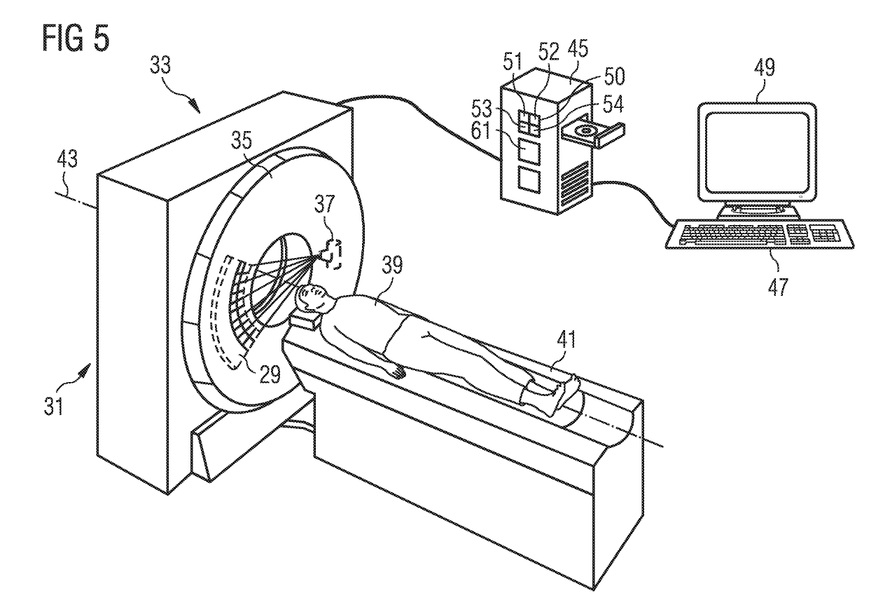

FIG. 5 shows an example embodiment of the computed tomography system 31 according to the invention for the execution of the method according to an example embodiment of the invention for imaging. The computed tomography system 31 contains a gantry 33 with a rotor 35. The rotor 35 includes an X-ray source 37 and the detector apparatus 29. The examination object 39 is supported on the patient bench 41 and can be moved along the axis of rotation z 43 through the gantry 33. The computed tomography system 31 further includes a computing unit 45.

An image reconstruction device 50 is used to control and calculate the image slices and to perform the method according to an example embodiment of the invention in particular for imaging. The image reconstruction 50 can include a generating unit 51, a selecting unit 52, a comparing unit 53 and an artifact correction unit 54. The image reconstruction device 50 can be included in the computing unit 45. An input device 47 and an output apparatus 49 are connected to the computing unit 45. The computing unit 45 can further include the training unit 61. A computer-readable data medium with program code of a computer program can be read by the computing unit 45 or included in the computing unit 45 in order to perform the method for imaging when the computer program is executed on a computer or the computing unit 45.

Although the invention was illustrated in more detail by the preferred example embodiment, the invention is not restricted by the disclosed examples and other variations can be derived herefrom by the person skilled in the art without departing from the scope of protection of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for imaging a region of interest of an object to be examined based on projection measurement data recorded during a rotational movement of an X-ray source-detector system around the object to be examined in a first angular sector of at least 180°, the method comprising:
generating first start-image data;
selecting partial projection measurement data with a second angular sector from the projection measurement data, the second angular sector being a subregion of the first angular sector;
comparing the first start-image data generated, or corrected start-image data, with the partial projection measurement data, and generating first image data based upon the comparing; and
artifact correcting the first image data via a trained correction unit, to create the corrected start-image data, the first image data and the corrected start-image data each respectively comprising a substantially complete image of the region of interest.

2. The method of claim 1, wherein the trained correction unit is based on a machine learning method, a statistical method, a mapping protocol, mathematical functions, an artificial neural network or a learning database.

3. The method of claim 1, wherein the generating includes generating the first start-image data based on the projection measurement data.

4. The method of claim 1, wherein the generating includes generating the first start-image data independent of the projection measurement data.

5. The method of claim 1, wherein the comparing and artifact correcting are performed iteratively, and wherein, initially the comparing includes comparing the first start-image data with the partial projection measurement data and, in at least one further comparing, the comparing includes comparing the corrected start-image data with the partial projection measurement data.

6. The method of claim 1, wherein the comparing is performed in an image space.

7. The method of claim 1, wherein the comparing is performed in a projection data space.

8. The method of claim 1, further comprising regularizationing.

9. The method of claim 1, wherein a second angular sector covers an angle of less than 180°.

10. The method of claim 9, wherein a second angular sector covers an angle in a range of 100° to 140°.

11. The method of claim 1, further comprising:
outputting the first image data or the corrected start-image data as a result image.

12. The method of claim 1, wherein the artifact correcting includes correcting an anatomical subregion of the object to be examined in the first image data.

13. An image reconstruction device, comprising:
a generating unit to generate first start-image data;
a selecting unit to select partial projection measurement data with a second angular sector from the projection measurement data, the second angular sector being a subregion of a first angular sector of a rotational movement of an X-ray source-detector system around the object to be examined;
a comparing unit to compare the first start-image data or corrected start-image data with the partial projection measurement data, and to generate first image data based upon the compare of the comparing unit; and
an artifact correction unit to artifact correct the first image data via a trained correction unit, to create the corrected start-image data.

14. A computed tomography system, comprising:
the image reconstruction device of claim 13.

15. A non-transitory memory storing program code for performing the method of claim 1 when the program code is executed on a computer.

16. A non-transitory computer-readable data medium storing program code of a computer program for performing the method of claim 1, when the computer program is executed on a computer.

17. A non-transitory computer-readable data medium storing program code of a computer program for performing the method of claim 2, when the computer program is executed on a computer.

18. The method of claim 2, wherein the generating includes generating the first start-image data based on the projection measurement data.

19. The method of claim 2, wherein the generating includes generating the first start-image data independent of the projection measurement data.

20. An image reconstruction device, comprising:
at least one processor configured to
generate first start-image data,
select partial projection measurement data with a second angular sector from the projection measurement data, the second angular sector being a subregion of the first angular sector of a rotational movement of an X-ray source-detector system around the object to be examined,
compare the first start-image data or corrected start-image data with the partial projection measurement data, and to generate first image data based upon the compare, and
artifact correct the first image data via a trained correction unit, to create the corrected start-image data.

21. A computed tomography system, comprising:
the image reconstruction device of claim 20.
